(19) United States
(12) Patent Application Publication
(10) Pub. No.: US 2023/0404749 A1
(43) Pub. Date: Dec. 21, 2023

(54) SYSTEMS AND METHODS FOR MANUFACTURING PROSTHETIC HEART VALVES USING A SINGLE-PIECE VALVE SUBASSEMBLY

(71) Applicant: Medtronic, Inc., Minneapolis, MN (US)

(72) Inventors: Victoria Tien, Apple Valley, MN (US); Yogesh A. Darekar, Tustin, CA (US); William Seo, Anaheim, CA (US); Genaro Morones, Jr., San Antonio, TX (US); Karl L. Olney, Irvine, CA (US); Andrew Tiem-Yen Chang, Torrance, CA (US); Veronica Woen, Westminster, CA (US); Michael Allen Darwin, San Francisco, CA (US); Marina Zamudio I Domingo, Irvine, CA (US); Garrett Repp, Gilbert, AZ (US)

(21) Appl. No.: 18/035,913

(22) PCT Filed: Nov. 8, 2021
(86) PCT No.: PCT/US2021/058389

§ 371 Date: May 8, 2023

Related U.S. Application Data
(60) Provisional application No. 63/111,328, filed on Nov. 9, 2020.

(51) Int. Cl. A61F 2/24; A61L 27/36

(52) U.S. Cl. CPC A61F 2/2415; A61F 2/2418; A61L 27/3641; A61F 2220/0075; A61F 2240/004

(57) ABSTRACT

A method of manufacturing a heart valve prosthesis includes cutting a flat sheet of a valve material into a single-piece valve pattern having two or more valve leaflet regions and a valve skirt region. The method also includes arranging the two or more valve leaflet regions of the single-piece valve pattern into a mold for forming a leaflet belly for each of the two or more valve leaflet regions. Further, the method includes fixing, while arranged in the mold, a shape of the leaflet belly of each of two or more valve leaflet regions of the single piece-valve pattern. The method additionally includes creating a side seam by attaching two longitudinal edges of the single-piece valve pattern, to thereby form a tubular valve subassembly with two or more valve leaflets. The method includes attaching the tubular valve subassembly within a tubular frame to form a heart valve prosthesis.

### FIELD

The present technology is generally related to medical devices. And, more particularly, to systems and methods for manufacturing prosthetic heart valves.

### BACKGROUND

Patients suffering from various medical conditions or diseases may require surgery to install an implantable medical device. For example, valve regurgitation or stenotic calcification of leaflets of a heart valve may be treated with a prosthetic heart valve. The prosthetic heart valve can be implanted at an implant site using traditional surgical procedures. As an alternative to a traditional surgical procedure, the prosthetic heart valve can be implanted using minimally-invasive techniques, for example, percutaneous and transluminal delivery to and deployment at an implant site.

A prosthetic heart valve typically includes a frame or stent that supports a heart valve structure that replaces the native heart valve. Conventionally, the heart valve structure is fabricated or manufactured by individually processing each component of the heart valve structure and individually assembling each component to form the heart valve structure. For example, a typical heart valve structure may include seven pieces of tissue, e.g., three valve leaflets, three skirts, and one wrap, that are individually processed and then hand sewn together to form the heart valve structure. Then, the heart valve structure is attached to the frame or stent by hand sewing the heart valve structure to the frame or stent.

This conventional process of fabricating the heart valve structure is time intensive and laborious. For instance, in the above example, it may require ten hours to assemble the heart valve structure and then attach it to a frame or stent. More particularly, it may take four hours to sew the pieces of the heart valve assembly together into a sub-assembly and an additional six hours to attach the sub-assembly onto the frame or stent. Due to the small size of the heart valve structure and the frame, the hand sewing requires highly trained and skilled workers to properly construct the heart valve prosthesis.

Accordingly, it is desirable to reduce the labor and time required to fabricate a valve structure and manufacture a prosthetic heart valve.

### SUMMARY

The techniques of this disclosure generally relate to systems and methods for manufacturing a prosthetic heart valve using a single-piece valve pattern and single-piece valve subassembly formed therefrom.

In one aspect, the present disclosure provides a method of manufacturing a heart valve prosthesis for transcatheter delivery. The method includes cutting a flat sheet of a valve material into a single-piece valve pattern having two or more valve leaflet regions and a valve skirt region. The method also includes arranging the two or more valve leaflet regions of the single-piece valve pattern into a mold for forming a leaflet belly for each of the two or more valve leaflet regions. Further, the method includes fixing, while arranged in the mold, a shape of the leaflet belly of each of two or more valve leaflet regions of the single piece-valve pattern. The method additionally includes creating a side seam by attaching two longitudinal edges of the single-piece valve pattern, to thereby form a tubular valve subassembly with two or more valve leaflets. The method includes attaching the tubular valve subassembly within a tubular frame to form a heart valve prosthesis.

In another aspect, the present disclosure provides a heart valve prosthesis. The heart valve prosthesis includes a tubular valve subassembly formed from a single-piece of valve material. The single-piece of valve material defining two or more valve leaflets and a valve skirt. The heart valve prosthesis also includes a tubular frame. The tubular valve assembly is secured within an interior of the tubular frame.

In another aspect, the present disclosure provides a system for forming a heart valve prosthesis. The system includes a cutting apparatus configured to cut a flat sheet of a valve material into a single-piece valve pattern having two or more valve leaflet regions and a valve skirt region. The system also includes a molding apparatus configured to form a leaflet belly for each of the two or more valve leaflet regions. The molding apparatus comprises at least two mating recesses and projections that form the valve material into a shape of the leaflet belly. Further, the system includes a folding apparatus configured to hold folds in the flat sheet of the valve material in each of the two or more valve leaflet regions while the cutting apparatus cuts the flat sheet of the valve material.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIG. 1 depicts an illustration of a valve manufacturing system, according to an embodiment hereof.

FIGS. 2A-2E depict several illustrations of a single-piece valve pattern that may be manufacturing using the valve manufacturing system of FIG. 1, according to an embodiment hereof.

FIGS. 3A-3C depict several illustrations of a folding device that may be used with the valve manufacturing system of FIG. 1, according to an embodiment hereof.

FIGS. 4A-4C depict several illustrations of another folding device that may be used with the valve manufacturing system of FIG. 1, according to an embodiment hereof.

FIGS. 5A-5C depict several illustrations of a molding apparatus that may be used with the valve manufacturing system of FIG. 1, according to an embodiment hereof.

FIG. 6 and FIGS. 7A-7E depict a process of manufacturing a prosthetic heart valve, according to an embodiment hereof.

FIG. 8 depicts another process of manufacturing a prosthetic heart valve, according to an embodiment hereof.

FIG. 9 depicts an illustration of a tubular frame that may be used with a valve structure, according to an embodiment hereof.

### DETAILED DESCRIPTION

Specific embodiments of the present disclosure are now described with reference to the figures. The following detailed description describes examples of embodiments and is not intended to limit the present technology or the application and uses of the present technology. Although the description of embodiments hereof is in the context of prosthetic heart valves, the present technology may also be used in other valve devices. Furthermore, there is no intention to be bound by any expressed or implied theory presented in the preceding technical field, background, brief summary or the following detailed description.

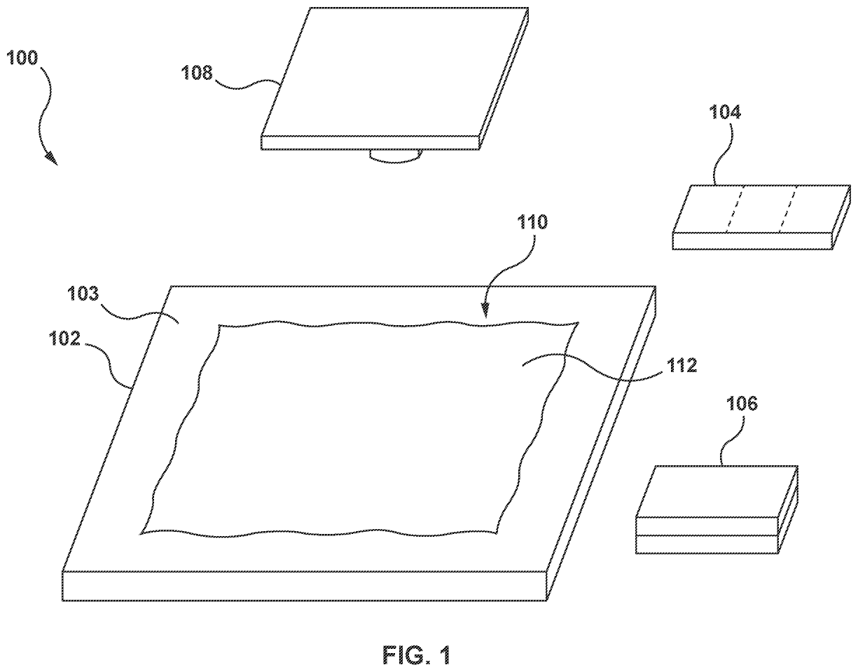

FIG. 1 illustrates a simplified schematic of a valve manufacturing system 100 in accordance with an embodiment hereof. One skilled in the art will realize that FIG. 1 illustrates one example of a valve manufacturing system and that existing components illustrated in FIG. 1 may be removed and/or additional components may be added to the valve manufacturing system 100.

As illustrated in FIG. 1, the valve manufacturing system 100 includes a work surface 102, a folding apparatus 104, a molding apparatus 106, and a cutting apparatus 108. The work surface 102 includes a substantially flat top surface area 103 that allows a flat sheet 110 of a valve material 110 to be spread out and cut into a single-piece valve pattern using the cutting apparatus 108. The single-piece valve pattern, after being fabricated, can be formed into a tubular valve subassembly, and coupled to a frame or stent to form a valve structure of a heart valve prothesis. As described herein, a single-piece valve pattern is a pattern that is formed of a single, continuous piece of the valve material 110.

The work surface 102 can be any suitable flat surface and may be constructed from metallic materials, such as stainless steel, or from plastic materials, such as acrylic, high density polyethylene, polypropylene, polyesters, polyamides and other suitable plastic materials. The work surface 102 can be constructed to dimensions, e.g., length and width, that allow the flat sheet 110 of the valve material 112 to be positioned substantially flat on the top surface area 103 of the work surface 102 during the fabrication of the single-piece valve pattern.

In embodiments, the valve material 112 can be any type of biological and/or synthetic material that can be used to fabricate and manufacture the single-piece valve pattern, which forms a valve structure for a heart valve prosthesis. For example, the valve material 112 can be a biological membrane, such as a mammalian tissue, of the type that can be obtained from humans, pigs, cows, and sheep, for example. Examples of mammalian tissue that can be used for the valve material 112 include porcine aortic root tissue, porcine pericardial tissue, equine pericardial tissue, and/or bovine pericardial tissue. Examples of synthetic materials include polyesters, Teflon, fluoropolymers, woven or knitted cloth, a heat setting fabrics, etc.

In embodiments, the cutting apparatus 108 can be positioned above the top surface area 103. The cutting apparatus 108 can be any type of device that cuts the flat sheet 110 of the valve material 112 into a single-piece valve pattern, which is used to form the valve structure. In some embodiments, the cutting apparatus 108 can include a laser that utilizes radiant energy to cut the flat sheet 110 of the valve material 112 into the single-piece valve pattern. For example, the cutting apparatus 108 can include a carbon dioxide (CO₂) laser. In this embodiment, the cutting apparatus 108 can also include and/or be coupled to one or more computer systems that control the operation of the laser to cut the flat sheet 110 of the valve material 112 into the single-piece valve pattern. For example, the laser can include or be mounted on movable supports that are controlled by the one or more computer systems. That is, the one or more computer systems can provide control signals to the laser to activate the laser and move the laser according to one or more designs or templates corresponding to the single-piece valve pattern, which are stored on storage devices of the one or more computer systems. While the cutting apparatus 108 is described as including a laser, in other embodiments a suitable cutting device may include one or more other cutting apparatus, for example, punches, cutting dies, saws, blades, etc.

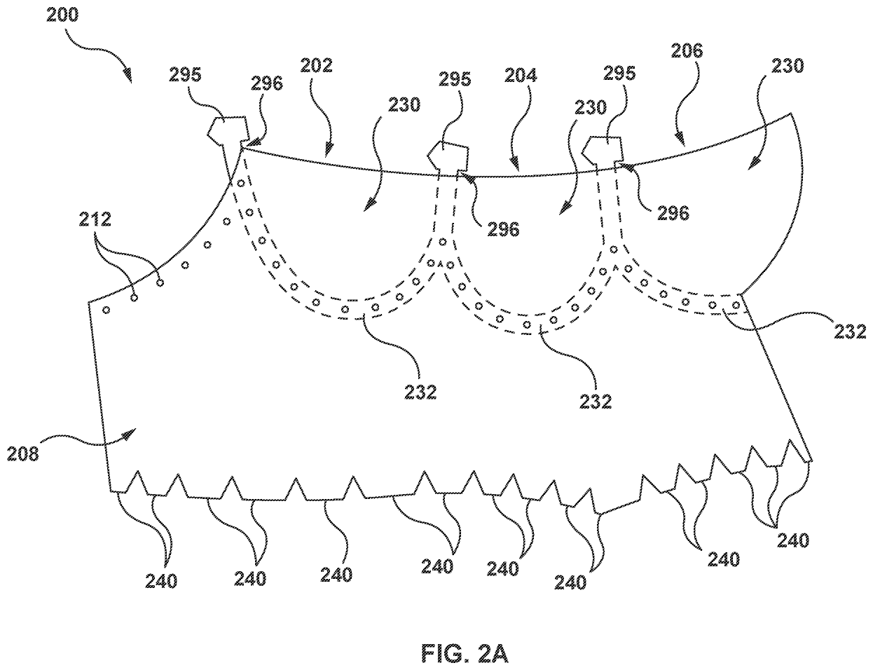

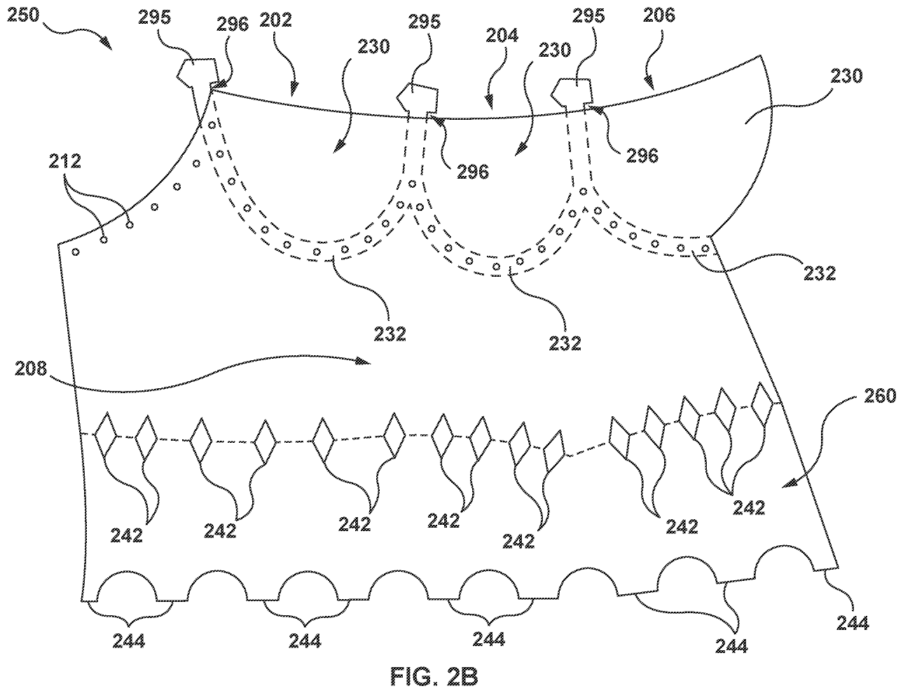

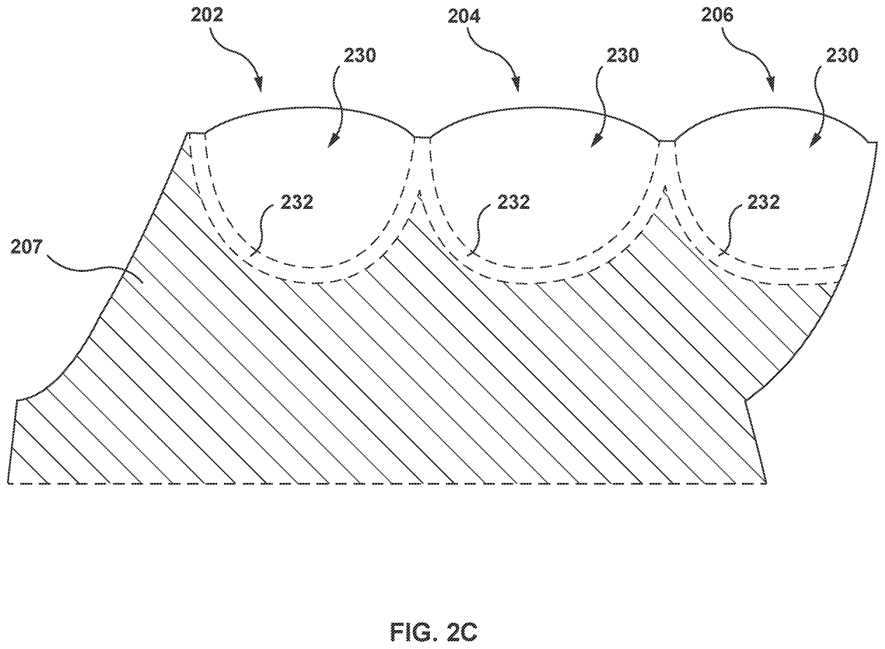

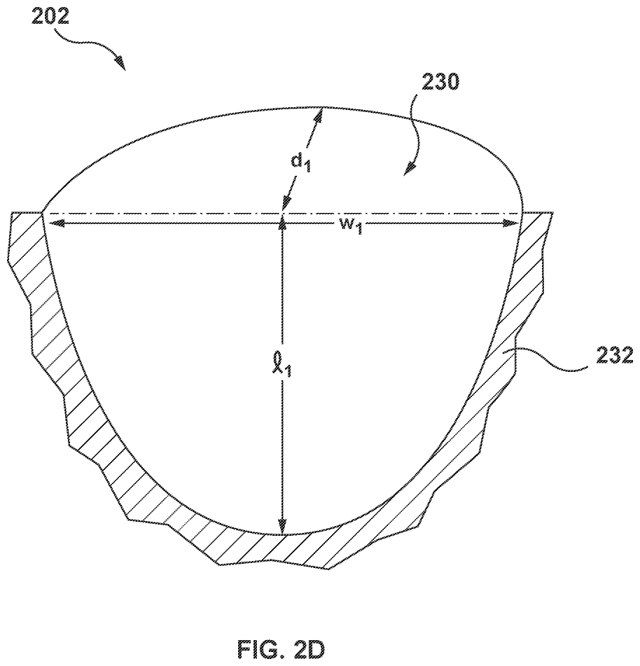

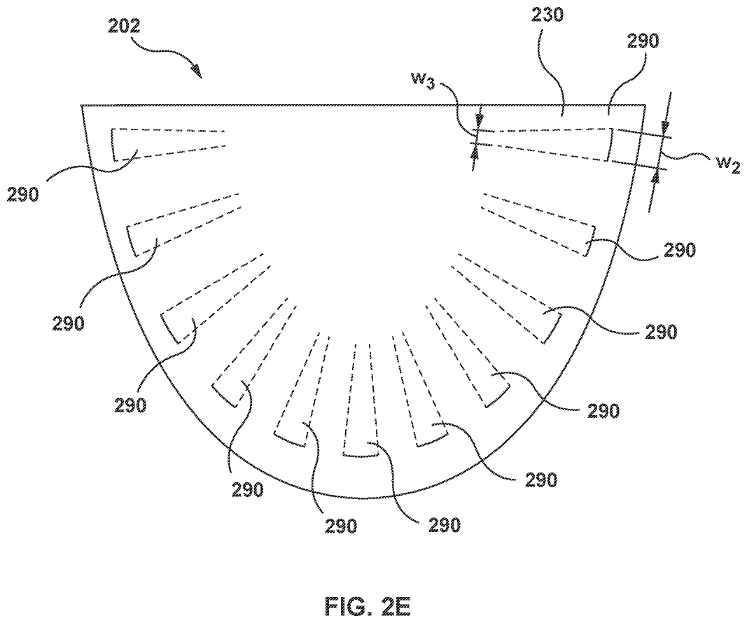

In embodiments, as described in further detail below, the single-piece valve pattern is a single and continuous piece of the valve material 112 that has been cut to include multiple components of the valve structure, e.g., valve leaflet regions, valve skirt regions, and/or an outer wrap region. In some embodiments, a single-piece valve pattern is a single piece of the valve material that includes two or more valve leaflet regions and valve skirt regions. In some embodiments, the single-piece valve pattern can include two or more valve leaflet regions, valve skirt regions, and an outer wrap region. FIGS. 2A-2E illustrate examples of single-piece valve patterns that may be cut from a flat sheet 110 of a valve material 112 in accordance with an embodiment hereof.

As illustrated in FIG. 2A, which is a top view, a single piece-valve pattern 200 may be cut from a flat sheet 110 of a valve material 112 to include three valve leaflet regions: a valve leaflet region 202, a valve leaflet region 204, and a valve leaflet region 206 (illustrated by dashes in FIG. 2A.) Additionally, the single piece-valve pattern 200 may be cut from the flat sheet 110 of the valve material 112 to include a skirt region 208 below the valve leaflet regions 202, 204, 206. In embodiments, the valve leaflet regions 202, 204, 206 form the three leaflets of the valve structure when the single-piece valve pattern 200 is assembled and attached to a frame or stent, as described below in further detail. The skirt region 208 forms a skirt of the valve structure when the single-piece valve pattern 200 is assembled and attached to a frame or stent.

In embodiments, the single piece-valve pattern 200 can be cut from the flat sheet 110 of the valve material 112 to include suture holes 212. The suture holes 212 can be positioned within an attachment margin 232 of each of the valve leaflet regions 202, 204, and 206. The suture holes 212 can provide a via through which sutures can be placed to attach the valve structure to a frame or stent. The skirt region 208 can be cut to include attachment tabs 240 along an edge of the skirt region that is opposite the valve leaflet regions. The attachment tabs 240 can be utilized to attach the valve structure formed from the single piece-valve pattern 200 to a corresponding end of a frame or stent.

As illustrated in FIG. 2B, which is a top view, similar to the single piece-valve pattern 200, a single piece-valve pattern 250 can be cut from a flat sheet 110 of a valve material 112 to include a valve leaflet region 202, a valve leaflet region 204, a valve leaflet region 206, and skirt region 208 (demarcated by dashes in FIG. 2B). Additionally, the single piece-valve pattern 250 can be cut from the flat sheet 110 of the valve material 112 to include an outer wrap region 260.

In embodiments, the single-piece valve pattern 250 can be cut from the flat sheet 110 of the valve material 112 to include the suture holes 212. The suture holes 212 can be positioned within the attachment margin 232 of each of the leaflet regions 202, 204, and 206. The single-piece valve pattern 250 can be cut to include attachment openings 242 formed along a perimeter between the skirt region 208 and the outer wrap region 260. The single-piece valve pattern 250 can also be cut to include outer attachment tabs 244 formed along a bottom perimeter of the outer wrap region 260. When the single-piece valve pattern 250 is assembled as a valve subassembly and attached to a frame, the attachment openings 242 wrap over an edge of the frame, where the valve leaflet regions 202, 204, 206 and skirt region 208 is located in the interior of the frame and the outer wrap region 260 is located on the exterior of the frame, and allow one or more components of the frame to extend through the attachment openings 242. The outer wrap region 260 can be attached to the frame by the outer attachment tabs 244.

As described below in further detail, when formed into a valve structure, the single-piece valve patterns 200 and 250 are assembled into a tubular valve subassembly to be attached to a frame or stent having a tubular shape (hereinafter “tubular frame.”) For the single-piece valve pattern 200, the tubular valve subassembly is attached to the tubular frame such that the tubular valve subassembly is positioned within the interior of the tubular frame, as described below in further detail. For the single-piece valve pattern 250, the tubular valve subassembly is attached to the tubular frame such that a portion of the tubular valve subassembly including leaflet regions 202, 204, 206 and the skirt region 208 is positioned within the interior of the tubular frame and the outer wrap region 260 is positioned around an outer surface of the exterior of the tubular frame, as described below in further detail.

In the single-piece valve patterns 200 and 250 described above, each leaflet regions 202, 204, 206 includes a leaflet belly 230, which is a bulge or depression formed in the valve material to extend along at least a portion of an attachment margin 232. Each leaflet belly 230 becomes a portion of a leaflet of the valve structure of a prosthetic heart valve. For the single-piece valve patterns 200 and 250, the leaflet regions 202, 204, 206 form three leaflets for a tricuspid valve structure of a prosthetic heart valve. While FIGS. 2A and 2B illustrate the single-piece valve patterns including three leaflet regions, a single piece valve pattern can include fewer leaflet regions, e.g., two leaflet regions forming two leaflets for a bicuspid valve structure, or alternatively additional leaflet regions may be added.

In some embodiments, as illustrated in FIGS. 2A and 2B, the single-piece valve patterns 200 and 250 can include ears 295. The ears 295 can be positioned at commissures 296 between the leaflet regions 202, 204, 206 and extend from a top surface of the single-piece valve patterns 200 and 250. As described below in further detail, once the single-piece valve patterns 200 and 250 are assembled into the tubular valve subassembly, the ears 295 can be utilized to attach the tubular valve subassembly to the tubular frame. In some embodiments, the ears 296 can be formed when constructing the single-piece valve patterns 200 and 250, for example, laser cut from the flat sheet 110 of the valve material 112. In some embodiments, the ears 296 can be formed separately and attached to the single-piece valve patterns 200 and 250, for example, by sutures. In some embodiment, the ears 296 can be omitted and the tubular valve subassembly can be attached to the tubular frame at the commissures 296, as described below in further detail.

As illustrated in FIG. 2C, which is a simplified perspective view of the leaflet regions and the skirt region, the single piece-valve pattern 200 and/250 has a flat or two-dimensional (2D) profile relative to atop planer surface 207 of the single piece-valve pattern 200 and/or 250. When formed into the tubular valve subassembly, the flat profile of the top planer surface 207 forms a continuous tubular surface for attachment to the inner surfaces of the tubular frame, as described below in further detail. When the tubular valve subassembly is attached to the tubular frame, the leaflets formed by the leaflet regions 202, 204, and 206 and their respective leaflet bellies 230 extend into the interior of the tubular frame toward a longitudinal axis thereof. As such, the leaflet belly 230 of each of the valve leaflet regions 202, 204, and 206 is formed to have a three-dimensional (3D) profile relative to the top planar surface 207 of the single piece-valve pattern 200 and/or 250.

For example, as illustrated in FIG. 2D, which is an enlarged perspective view of the leaflet region 202, the leaflet belly 230 is formed having 3D half-bell or paraboloid shape. Dimensions of the 3D paraboloid shape of the leaflet belly 230 can be defined by a maximum width, w₁, a maximum length l₁, and a maximum depth d₁. The dimensions can be dependent on the size of the heart valve prosthesis that includes the single piece-valve pattern 200 and/250, e.g., a 23 millimeter (mm) heart valve prosthesis, a 26 mm heart valve prosthesis, a 29 mm heart valve prosthesis, 34 mm heart valve prosthesis, etc. For example, in a 29 mm heart valve prosthesis, the dimensions for leaflet belly 230 may include: a maximum width, w₁, of approximately 22 mm, a maximum length, l₁, of approximately 16 mm, and a maximum depth, d₁, of approximately 5 mm. One skilled in the art will realize that any examples of dimensions describe herein are approximate values and can vary by, for example, +/−5.0%, based on manufacturing tolerances, operating conditions, and/or other factors.

As discussed above, when cutting the single-piece valve pattern 200 and/or 250, a flat sheet 110 of a valve material 112 is spread out of a work surface 102, e.g., laid flat. Because a leaflet belly 230 of each of the leaflet regions 202, 204, 206 needs to be formed having a 3D shape, extra material needs to be gathered in the leaflet regions 202, 204, 206 prior to cutting by the cutting apparatus 108. In embodiments, as illustrated in FIG. 2E which is a simplified view of the leaflet region 202, additional valve material 112 can be gathered in the leaflet regions 202, 204, 206 by forming triangular pleats or folds 290 (hereinafter triangular folds) of the valve material 112 in the leaflet regions 202, 204, 206. In embodiments, the triangle folds 290 include extra material that has been gathered relative to the flat sheet 110 of the valve material 112. In embodiments, as illustrated in FIG. 2E, the triangular folds 290 have a triangular shape such that a width, w₂, adjacent to the attachment margin 232 is larger than a width, w₃, adjacent to a center of the leaflet regions 202, 204, 206. As such, an amount of valve material gathered in the triangular folds 290 decreases from the attachment margin 232 to the center of the leaflet belly 230. For example, the dimensions of triangular folds 290 may include a width, w₂, ranging from approximately 1 mm to approximately 2 mm, and a width, w₃, ranging from approximately mm to approximately 0.2 mm.

In some embodiments, the triangular folds 290 can be formed by sewing the valve material 112 into pleats to form the triangular folds 290 in each of the valve leaflet regions 202, 204, 206, for example, cinching the valve material 112 in the valve leaflet regions 202, 204, 206 with a suture to create pleats. In some embodiments, the triangular folds 290 can be formed using the folding apparatus 104 prior to cutting the single-piece valve pattern 200 and/or 250 with the cutting apparatus 108.

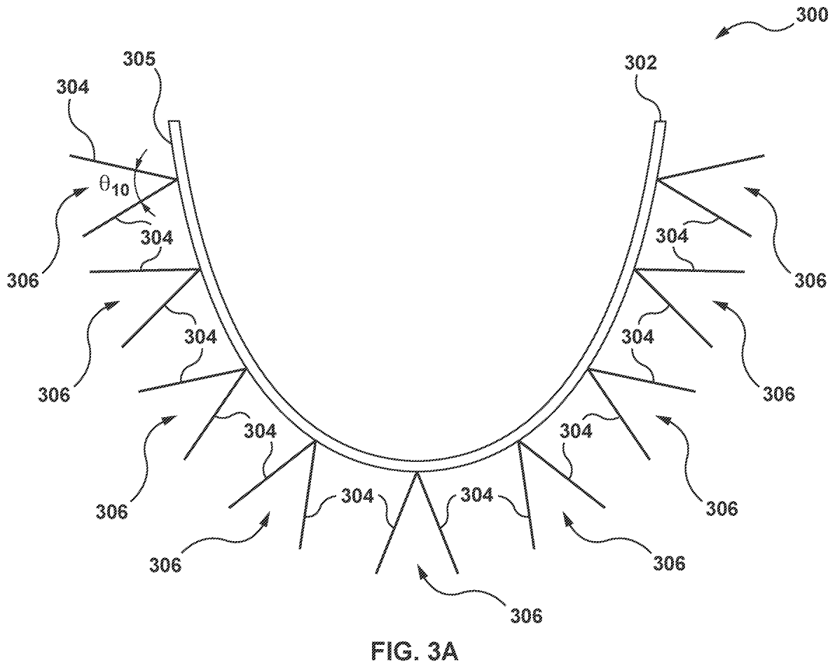

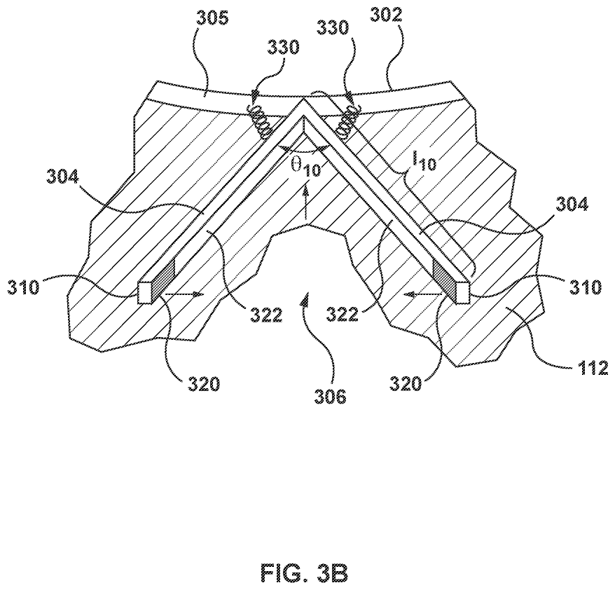

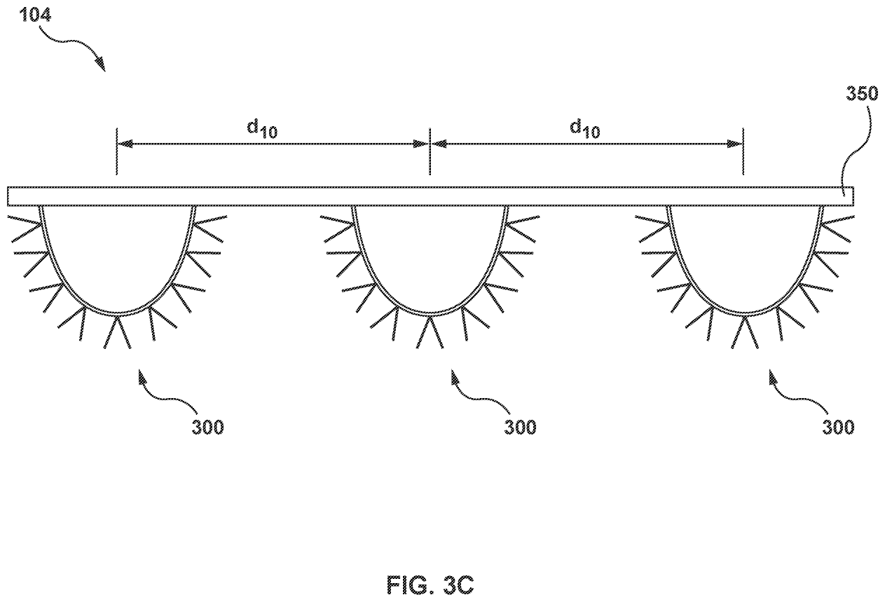

FIGS. 3A-3C are several views of a folding device 300 in accordance with an embodiment hereof. One skilled in the art will realize that FIGS. 3A-3C illustrate one example of a folding device and that existing components illustrated in FIGS. 3A-3C may be removed and/or additional components may be added to the folding device 300.

As illustrated in FIG. 3A, the folding device 300 includes a u-ring 302 and arms 304 attached to an outer surface 305 of the u-ring 302. An adjacent pair of the arms 304 defines a folding space 306 in which the triangular folds 290 can be formed. That is, adjacent pairs of the arms 304 extend outward from the u-ring 302 at an angle, θ₁₀, relative to one another. The folding device 300 can include a number of adjacent pairs of the arms 304 corresponding to the number of triangular folds 290 to be formed in the leaflet regions 202, 204, 206.

As illustrated in FIG. 3B, which is an enlarged perspective view of an adjacent pair of arms 304, the arms 304 extend outward from the outer surface 305 of the u-ring 302 at the angle, θ₁₀, relative to one another. The arms 304 can be formed having a length, ho, from a connection to the outer surface 305 of the u-ring 302 to an end 310 of the arms 304. In some embodiments, the length, l₁₀, can correspond to an approximate length of the triangular folds 290. The length, l₁₀, can depend on the size of the leaflets being formed from the leaflet regions 202, 204, 206. For example, the arms 304 may be formed having a length, l₁₀, that ranges from approximately 10 mm to approximately 16 mm.

Each of the arms 304 includes a clamp 320 coupled to an end 310 of an arm 304. The clamps 320 can be constructed of a material that provides a higher coefficient of friction relative to the arms 304. In an embodiment, the clamps 320 can extend from the ends 310 of the arms 304 on an inner surface 322 of the arms 304 that faces the folding space 306. As such, the clamps 320 can operate to grip the valve material positioned in the folding space 306 when the arms 304 are biased inward towards one another.

The arms 304 suitably are constructed of a resilient material that permits the arms 304 to be separated, biased inward towards one another. For example, the arms 304 can be constructed of a flexible metal, flexible metal alloy, flexible polymeric material, shape memory metal alloys (e.g., nitinol), etc. The clamp 320 operates to bias the arms 304 together at the ends 310 to trap, clamp and/or hold valve material therebetween. In an embodiment, biasing members 330 can be coupled to the arms 304. The biasing members 330 can be configured to exert a force on the arms 304 to bias the arms inwards towards one another thereby reducing the folding space 306. For example, the biasing members 330 can include one or more springs that are coupled between the arms 304 and the u-ring 302. The springs can exert a force on the arms 304 inward towards the folding space 306 thereby causing the arms 304 to exert a force on valve material positioned in the folding space 306.

As further illustrated in FIG. 3B, to create triangular folds 290, the folding device 300 can be placed on a top surface of the flat sheet 110 of the valve material 112. The valve material 110 can then be pulled upward through each folding space 306 formed by each adjacent pair of the arms 304. Because adjacent pairs of the arms 304 are positioned at the angle, θ₁₀, relative to one another, more material can be pulled upward though the folding space 306 at the ends 310, thereby forming the triangular folds 290. The clamps 320 of adjacent pair of arms 304 then bias inward toward each other to clamp, pinch and/or hold the extra valve material 112 during cutting by the cutting apparatus 108.

In embodiments, a folding apparatus 104 of the valve manufacturing system 100 can include a number of the folding devices 300 that corresponds to the number of leaflet regions included in the single-piece valve pattern. For example, as illustrated in FIG. 3C, the folding apparatus 104 can include three folding devices 300 for creating the triangular folds 290 in the single-piece valve pattern 200 and/or 250. In the folding apparatus 104, the folding devices 300 can be coupled to a spacing bar 350. The folding devices 300 can be coupled to the spacing bar 350 at a distance, d₁₀, relative to one another. In some embodiments, the distance, d₁₀, can corresponding to the spacing distances of the leaflet regions 202, 204, 206 in the single-piece valve pattern 200 and/or 250. For example, the d distance, d₁₀, can correspond to the linear distance from a midpoint of adjacent folding devices 300 measured along the spacing bar 350. The distance, d₁₀, can be selected so that the folding devices 300 align with the leaflet regions 202, 204, 206 when a flat sheet 110 of the valve material 112 is placed on the work surface 102.

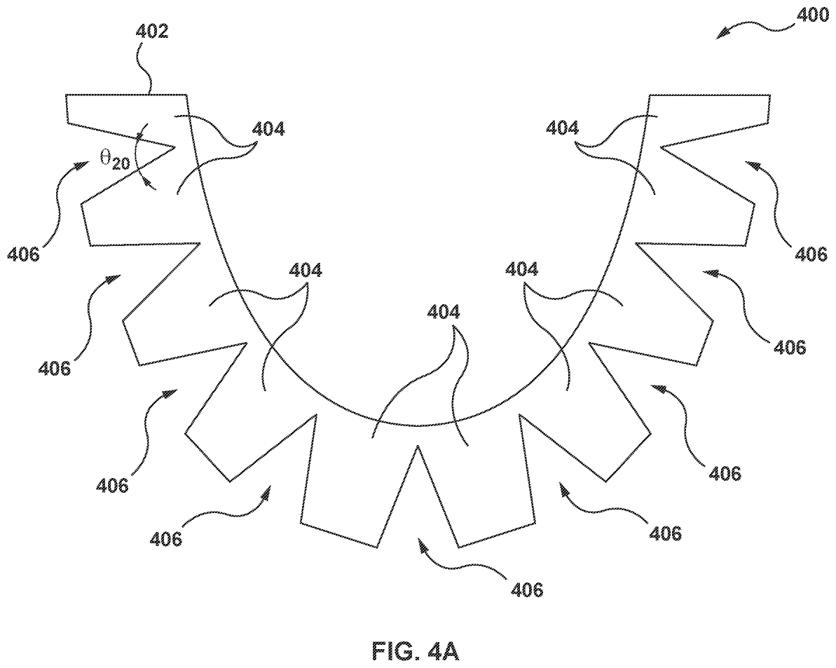

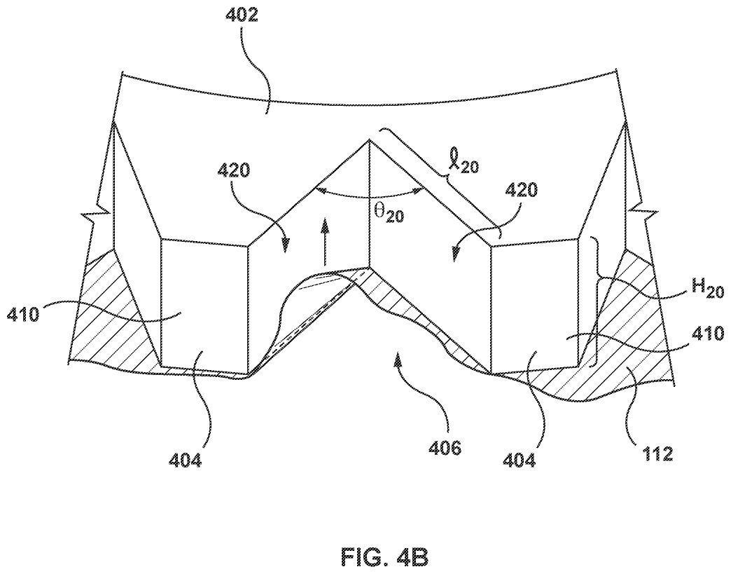

FIGS. 4A-4C are several views of a folding device 400 in accordance with an embodiment hereof. One skilled in the art will realize that FIGS. 4A-4C illustrates one example of a folding device and that existing components illustrated in FIGS. 4A-4C may be removed and/or additional components may be added to the folding device 400.

As illustrated in FIG. 4A, the folding device 400 includes a u-shaped body 402 and fixed tabs (or blocks) 404 extending outward from the u-shaped body 402. The fixed tabs 404 provide a rigid or semi-rigid structure that can be utilized to create folds in the flat sheet 110 of the valve material 112. In some embodiments, the fixed tabs 404 can be separate structures that are attached (removably or permanently) to the u-shaped body 402. In some embodiments, the fixed tabs 404 and the u-shaped body 404 can be constructed as a single integrated structure.

An adjacent pair of the fixed tabs 404 defines a folding space 406 in which the triangular folds 290 can be formed. That is, adjacent pairs of the fixed tabs 404 extend outward from the u-shaped body 402 at an angle, θ₂₀, relative to one another. The folding device 400 can include a number of adjacent pairs of the fixed tabs 404 corresponding to the number of triangular folds 290 to be formed in the leaflet regions 202, 204, 206.

As illustrated in FIG. 4B, which is an enlarged perspective view of an adjacent pair of fixed tabs 404, the fixed tabs 404 extend outward from the u-shaped body 402 at the angle, θ₂₀, relative to one another. The fixed tabs 404 can be formed having a length, l₂₀, to extend from a connection to the u-shaped body 402 to an end 410 of the fixed tabs 404. The fixed tabs 404 can be formed having a height, H₂₀. In some embodiments, the length, l₂₀, can correspond to an approximate length of the triangular folds 290. The dimensions of the fixed tabs 404 can depend on the size of the leaflets being formed from the leaflet regions 202, 204, 206. For example, the fixed tabs 404 can be formed having a length, l₂₀, that ranges from approximately 5 mm to approximately 20 mm, e.g., 10 mm. The fixed tabs 404 can be constructed of a rigid material that allows the fixed tabs 404 to stay fixed when valve material is pulled through the folding spaces 406. For example, the fixed tabs 404 can be constructed of a rigid metal, rigid metal alloy, rigid polymeric material, etc.

In an embodiment, a portion of inner surfaces 420 of the fixed tabs 404, which face the folding space 406, can include and/or be coated with a material that provides a higher coefficient of friction relative to the fixed tabs 404. As such, the material on a portion of the inner surfaces 420 can operate to grip the valve material positioned in the folding space 406.

To create triangular folds 290, the folding device 400 can be placed on top of the flat sheet 110 of the valve material 112, as further illustrated in FIG. 4B. The valve material 112 can then be pulled upward through each folding space 406 formed by each adjacent pair of the fixed tabs 404. Because adjacent pairs of the fixed tabs 404 are positioned at the angle, θ₂₀, relative to one another, more material can be pulled upward through the folding space 406 at the ends 410, thereby forming the triangular folds 290.

In embodiments, the folding apparatus 104 of the valve manufacturing system 100 can include a number of the folding devices 400 that corresponds to the number of leaflet regions included in the single-piece valve pattern. For example, as illustrated in FIG. 4C, the folding apparatus 104 can include three folding devices 400 for creating the triangular folds 290 in the single-piece valve pattern 200 and/or 250. In the folding apparatus 104, the folding devices 400 can be coupled to a spacing bar 450. The folding devices 400 can be coupled to the spacing bar 450 at a distance, d₂₀, relative to one another. In some embodiments, the distance, d₂₀, can corresponding to the spacing distances of the leaflet regions 202, 204, 206 in the single-piece valve pattern 200 and/or 250. For example, the d distance, d₂₀, can correspond to the linear distance from a midpoint of adjacent folding devices 400 measured along the spacing bar 450. The distance, d₂₀, can be selected so that the folding devices 400 align with the leaflet regions 202, 204, 206 when a flat sheet 110 of the valve material 112 is placed on the work surface 102.

While the folding devices 300 and 400 are described above as operating to create triangular folds 290, one skilled in the art will realize that the triangular shape of the folds 290 is one example of a shape of the folds 290. In embodiments, the folds 290 can be created having a different shape based on a desired shape of the leaflet regions 202, 204, 206.

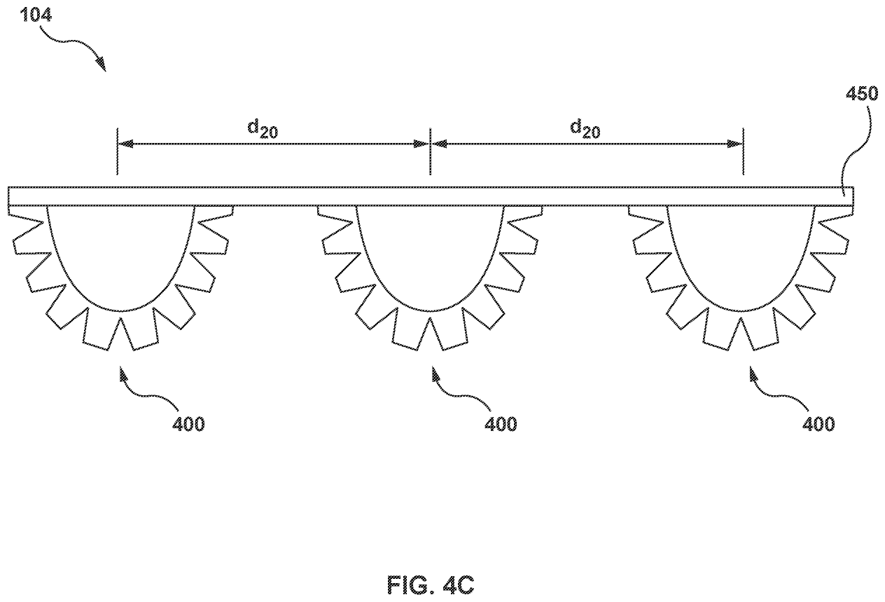

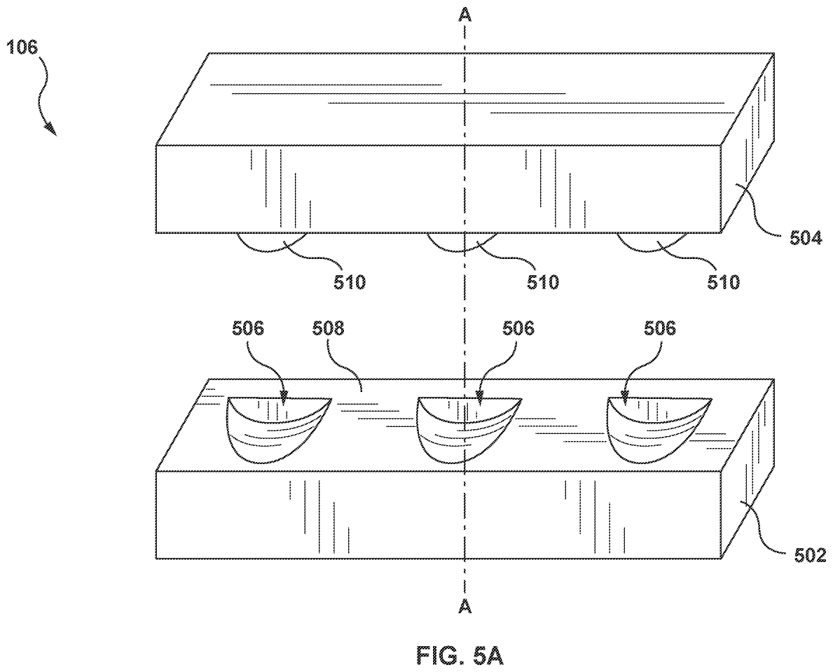

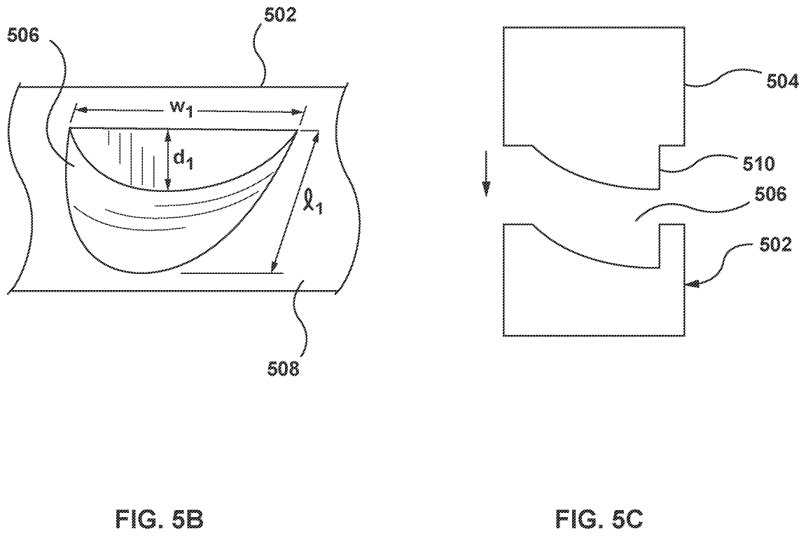

FIGS. 5A-5C illustrate a molding apparatus 106 for forming leaflet bellies of a valve structure in accordance with an embodiment hereof. One skilled in the art will realize that FIGS. 5A-5C illustrates one example of a molding apparatus and that existing components illustrated in FIGS. 5A-5C may be removed and/or additional components may be added to the molding apparatus 106.

As illustrated in FIG. 5A, the molding apparatus 106 includes a bottom mold 502 and a top mold 504. The bottom mold 502 includes a number of recesses 506 formed in a top surface 508 of the bottom mold 502. For example, for the single-piece valve pattern 200 and/or 250, the bottom mold 502 can include three recesses 506. Each of the recesses 506 is constructed having the 3D shape of a leaflet belly 230. As discussed above, the leaflet belly 230 of each of the valve leaflet regions 202, 204, and 206 is formed to have a 3D profile (shape) relative to the top planar surface 270 of the single piece-valve pattern 200 and/or 250. For example, as illustrated above in FIG. 2D, the leaflet belly 230 can be formed having 3D half-bell or paraboloid shape. As illustrated in FIG. 5B, which is an enlarged view of a recess 506, the recess 506 can have a 3D half-bell or paraboloid shape that corresponds to a desired 3D shape of a leaflet belly 230. The recess 506 is constructed having dimensions that correspond to the desired dimension of the leaflet belly 230. For example, as illustrated in FIG. 5B, the dimensions of the recess 506 can be defined by a maximum width, w₁, a maximum length l₁, and a maximum depth d₁.

The top mold 504 includes a number of projections 510 that extend from a bottom surface 512 of the top mold. As illustrated in FIG. 5C, which is a cross-section view taken along line A-A of FIG. 5A, each of the projections 510 has a 3D shape that mates with a shape of a recess 506 and is positioned on the bottom surface 512 to correspond with a position of a recess 506. During operations, the bottom mold 502 and the top mold 504 are brought together to compress a valve material between the mating recesses/molding surfaces 506 and projections/molding surfaces 510 thereby forming the leaflet bellies 230 in a 3D shape. As such, each of the projections 510 is constructed having dimensions that are smaller than the dimensions of a corresponding recess 506 thereby allowing each of the projections 510 to fit within a corresponding recess 506. Further operations of the molding apparatus 106 is discussed below in further details in the description of the method of manufacturing a prosthetic heart valve.

As noted above, the recesses 506 and the projections 510 are designed to form leaflet bellies 230 that are sized and shaped to be substantially similar to a corresponding functional native heart valve. For example, the recesses 506 and the projections 510 are sized, shaped and positioned to provide leaflet bellies 230, which when assembled, form leaflets that will desirably be sized and shaped so that they contact each other when the valve is closed to ensure proper valve closure. In embodiments, the recesses 506 and the projections 510 of varying sizes may be provided to accommodate the anatomies of patients with varying heart valve root annulus dimensions. In some embodiments, a molding apparatus 106 can include recesses 506 and projections 510 that are the same or different sizes and shapes. In some embodiments, a molding apparatus 106 can include recesses 506 and projections 510 that are fabricated to provide a heart valve that is designed for a specific patient.

While the molding apparatus 106 is described as having a bottom mold 502 with the recesses 506 and a top mold 504 with the projections 510, one skilled in the art will realize that the use of top and bottom are relative terms. In some embodiments, the bottom mold 502 can include the projections 510 and the top mold 504 can include the recesses 506.

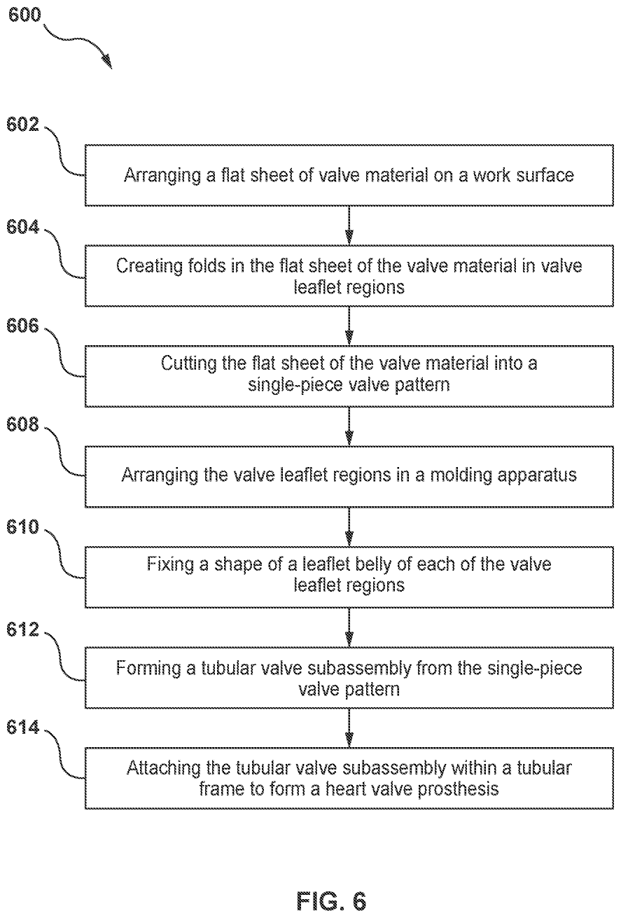

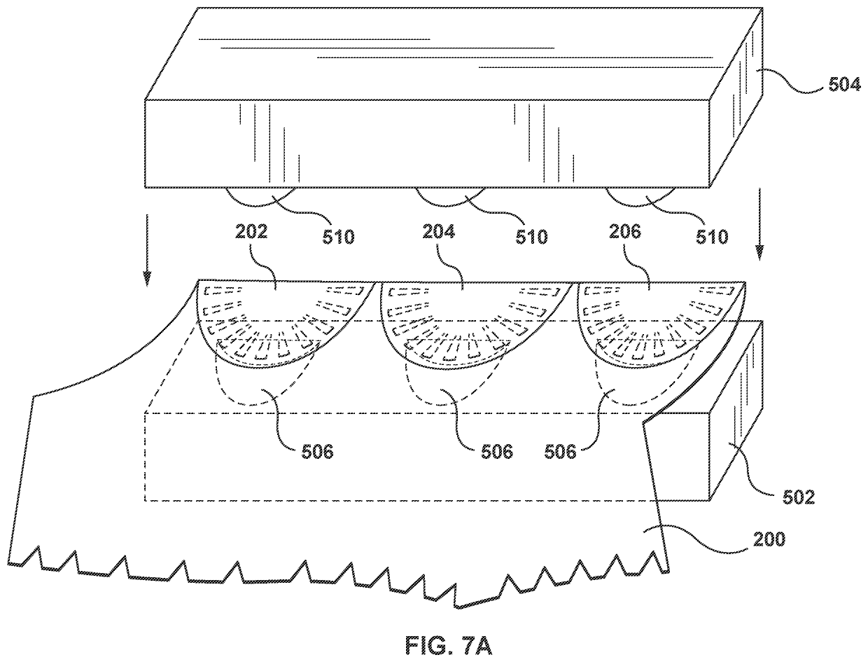

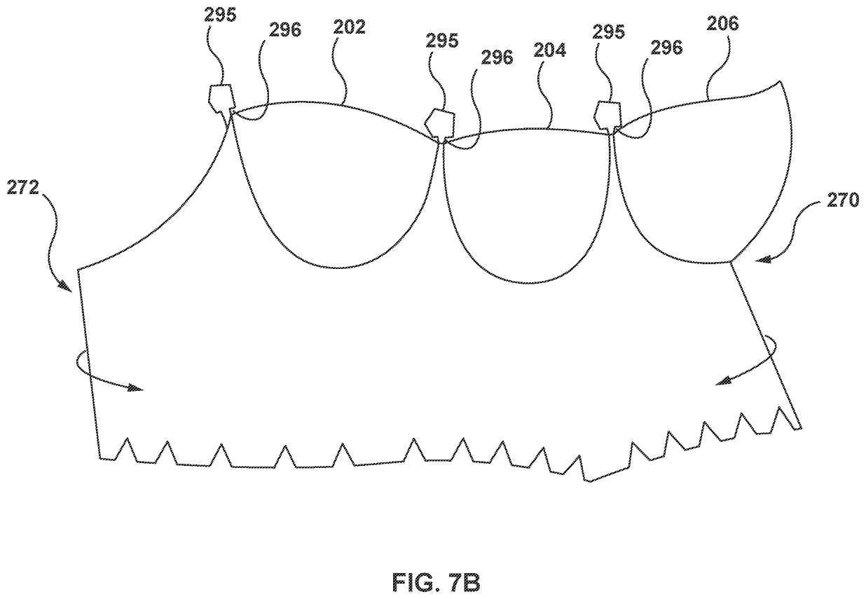

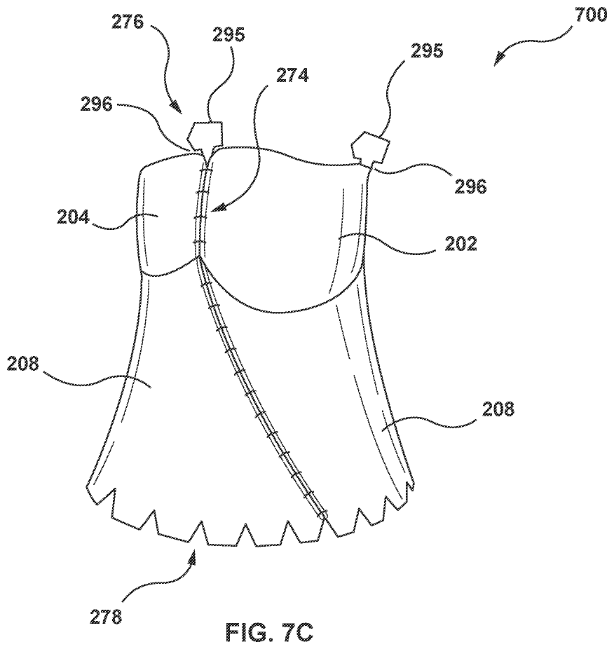

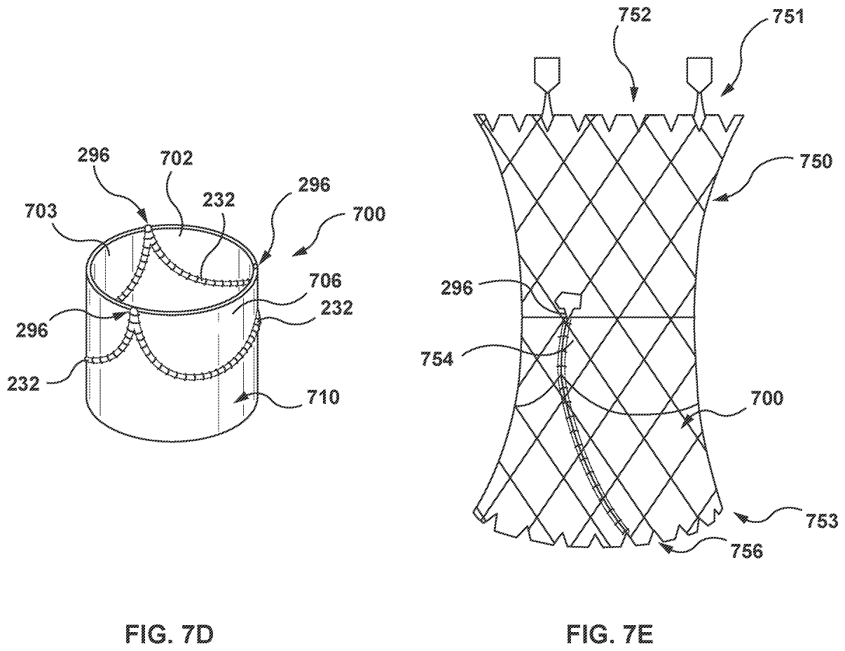

FIGS. 6 and 7A-7E illustrate an example of a method 600 for manufacturing a prosthetic heart valve, in accordance with an embodiment hereof. One skilled in the art will realize that FIGS. 6 and 7A-7E illustrate one example of a method using the manufacturing system 100 and that existing operations illustrated in FIG. 6 may be removed and/or additional operations may be added to the method 600.

In step 602, a flat sheet of valve material is arranged on a work surface. For example, as illustrated above in FIG. 1, a flat sheet 110 of a valve material 112 can be placed and arranged to be substantially flat on a top surface area 103 of a work surface 102.

In step 604, folds are created in the flat sheet of valve material in valve leaflet regions. In embodiments, once the flat sheet 110 of the valve material 112 is placed on the work surface 102, extra material, required to form the belly regions 230 needs to be gathered in the leaflet regions 202, 204, 206 prior to cutting by the cutting apparatus 108. Additional valve material 112 can be gathered in the leaflet regions 202, 204, 206 by forming triangular folds 290 of the valve material 112 in the leaflet regions 202, 204, 206, as illustrated above in FIG. 2E. The triangular folds 290 can be formed having a triangular shape such that a width, w₂, adjacent to the attachment margin 232 is larger than a width, w₃, adjacent to a center of the leaflet regions 202, 204, 206.

In some embodiments, the triangular folds 290 can be formed by sewing the valve material 112 into pleats to form the triangular folds 290 in each of the valve leaflet regions 202, 204, 206. For example, the triangular folds 290 can be hand sewn into the valve leaflet regions 202, 204, and 206.

In some embodiments, the triangular folds 290 can be formed using the folding apparatus 104 prior to cutting the single-piece valve pattern 200 and/or 250 with the cutting apparatus 108. For example, a folding apparatus 104 that includes folding devices 300 can be placed on the flat sheet 110 of the valve material 112 and arranged in the valve leaflet regions 202, 204, and 206. Once arranged, the valve material 110 can then be pulled upward through each folding space 306 formed by each adjacent pair of the arms 304 to be clamped into triangular folds 290 therebetween. The extra valve material 112 may be held within the arms 304 during cutting by the cutting apparatus 108.

Likewise, for example, a folding apparatus 104 that includes folding devices 400 can be placed on a flat sheet 110 of a valve material 112 (resting on the work surface 102) and arranged in the valve leaflet regions 202, 204, and 206. Once arranged, the valve material 110 can then be pulled through each folding space 406 formed by each adjacent pair of the fixed tabs 404 thereby forming the triangular folds 290.

In step 606, the flat sheet of the valve material is cut into a single-piece valve pattern. In embodiments, the cutting apparatus 108 can be activated to cut a single-piece valve pattern, such as the single-piece valve pattern 200 and/or 250. Because the valve leaflet regions 202, 204, and 206 includes the triangular folds 290, the single-piece valve pattern 200 and/or 250 includes extra valve material 112 required to from the leaflet bellies 230. Once the single-piece valve pattern 200 and/or 250 is cut, if used, the folding apparatus 104 can be removed from the single-piece valve pattern 200 and/or 250. Likewise, if the triangular fold 290 were sewn, the stiches can be removed from the single-piece valve pattern 200 and/or 250.

In step 608, the valve leaflet regions are arranged in a molding apparatus. The molding apparatus 106 operates to shape the extra valve material 112 formed by the triangular folds 290 into the leaflet bellies 230. As illustrated in FIG. 7A, the bottom mold 502 and the top mold 504 can be separated thereby exposing the recesses 506 and the projection 510. The single-piece valve pattern 200 and/or 250 can be arranged in the molding apparatus 106 such that the valve leaflet regions 202, 204, 206 align between a respective recess 506 and its mating projection 510. For example, each of the valve leaflet regions 202, 204, 206 can be arranged with a respective recess 506. Once arranged, the bottom mold 502 and the top mold 504 are brought together thereby capturing the valve leaflet regions 202, 204, 206 between the recesses 506 and the projections 510.

In step 610, a shape of a leaflet belly of each of the valve leaflet regions is fixed. In embodiments, to fix the shape of the leaflet bellies 230, the molding apparatus 106 is introduced to a fixing agent. For example, the molding apparatus 106 having the single-piece valve pattern 200 and/or 250 sandwiched between the bottom mold 502 and the top mold 504 is submerged in a fixation solution, such as a solution having glutaraldehyde, to cross-link the tissue. Cross-linking methods and solutions are well-known to the art. During submersion in the fixation solution, the solution percolates through the recesses 506 to cross-link the tissue. Then, the molding apparatus 106 is removed from the fixation solution, and rinsed.

In step 612, a tubular valve subassembly is formed from the single-piece valve pattern. In embodiments, the tubular valve subassembly is formed by forming the single piece valve pattern 200 and/or 250 into a tubular structure. For example, as illustrated in FIG. 7B, longitudinal edges 270 and 272 of the single piece valve pattern 200 and/or 250 can be brought together in a mating or otherwise abutting relationship. As illustrated in FIG. 7C, once mated, a side seam 274 can be created that runs from a first end 276 of the single piece valve pattern 200 and/or 250 to a second end 278 of the single piece valve pattern 200 and/or 250 thereby joining the longitudinal edges 270 and 272 and forming the tubular valve subassembly 700. In an embodiment, the side seam 274 is hand sewn with a suture or other filament.

As illustrated in FIGS. 7A and 7B, in some embodiments, the single-piece valve patterns 200 and/or 250 can include ears 295, positioned at the commissures 296 between the leaflet regions 202, 204, 206 and extending from a first end 276 of the single-piece valve patterns 200 and/or 250. In some embodiments, the ears 296 can be formed when constructing the single-piece valve patterns 200 and 250, for example, laser cut from the flat sheet 110 of the valve material 112 in step 606. In some embodiments, the ears 296 can be formed separately and attached to the single-piece valve patterns 200 and 250, for example, by sutures, at any point after step 606. In some embodiments, the ears 296 can be omitted, as illustrated in FIG. 7D.

In embodiment, prior to or after forming the completed tubular valve subassembly 700, additional processing can be performed to create, reinforce and/or modify certain aspects of the completed tubular valve assembly 700, as illustrated in FIG. 7D. In some embodiments, for example, a suture or other thread/filament may be overstitched along the attachment margin 232, such as within any suture holes 212 therein, to create, demarcate and/or reinforce each attachment margin 232 at a lower edge of each leaflet. In some embodiments, for example, the valve material at the attachment margin 232 can be folded and stitched using a suture or other thread/filament to create, demarcate and/or reinforce each attachment margin 232 at a lower edge of each leaflet. In some embodiments, for example, extra valve material can be attached to the attachment margin 232 using a suture or other thread/filament to create, demarcate and/or reinforce each attachment margin 232 at a lower edge of each leaflet.

Likewise, for example, commissures that are created at the juncture between adjacent valve leaflets formed by the valve leaflet regions 202, 204, 206 may be reinforced by sewing a suture or other thread/filament, in the suture holes 212 if utilized, at the junctures between adjacent leaflets. In some embodiments, for example, the ears 295, when formed separately from the single-piece valve patterns 200 and/or 250, can be added at the commissures 296 by sewing a suture or other thread/filament. The tubular valve subassembly 700, as illustrated in FIG. 7D, is formed from a single-piece of valve material that defines leaflets 702, 704, 706 (formed from the valve leaflet regions 202, 204, 206 and their leaflet bellies 230), commissures 296 and a valve skirt 710.

In step 614, the tubular valve subassembly 700 is attached within a tubular frame to form the heart valve prosthesis. As illustrated in FIG. 7E, the tubular valve subassembly 700 can be inserted into an interior or lumen 752 of a tubular frame 750 via either a first end 751 or a second end 753. Once inserted into the interior 752, the tubular valve subassembly 700 can be sewn onto the inner surfaces of the tubular frame 750, e.g., struts 754, using conventional techniques. For example, if the tubular valve subassembly 700 includes the ears 296, the ears 296 can be wrapped around a strut 754 and secured to the strut 754 using a suture or other thread/filament. In the above example, the tubular valve subassembly 700 extends along an entirety, or a near entirety, of a length of the tubular frame 750, but in other embodiments may extend less than the entire length of the tubular frame 750. A wide variety of other constructions are also acceptable and within the scope of the present disclosure.

For the tubular valve subassembly 700 constructed from the single-piece valve patterns 250 that includes the outer wrap region 260, a portion of the tubular valve subassembly 700 that includes the valve leaflet regions 202, 204, 206 and skirt regions 208 can be inserted into the interior or lumen 752 of a tubular frame 750 via the second end 753. Once inserted into the interior 752, the portion the tubular valve subassembly 700 that includes the valve leaflet regions 202, 204, 206 and skirt regions 208 can be sewn onto the inner surfaces of the tubular frame 750, e.g., struts 754, using conventional techniques, as discussed above. Once secured, the outer wrap region 260 can be folded over the second end 753 such that features of the tubular frame 750 can pass through the attachment opening 242 located between the outer wrap region 260 and the skirt region 208. For example, as the outer wrap region 260 is folded over the second end 753, crown 756 of the tubular frame 750 (or crowns 956 as described below in further detail) can pass through the attachment opening 242. Once folded, the outer wrap region 260 of the tubular valve subassembly 700 can be sewn onto the outer surfaces of the tubular frame 750 using conventional techniques.

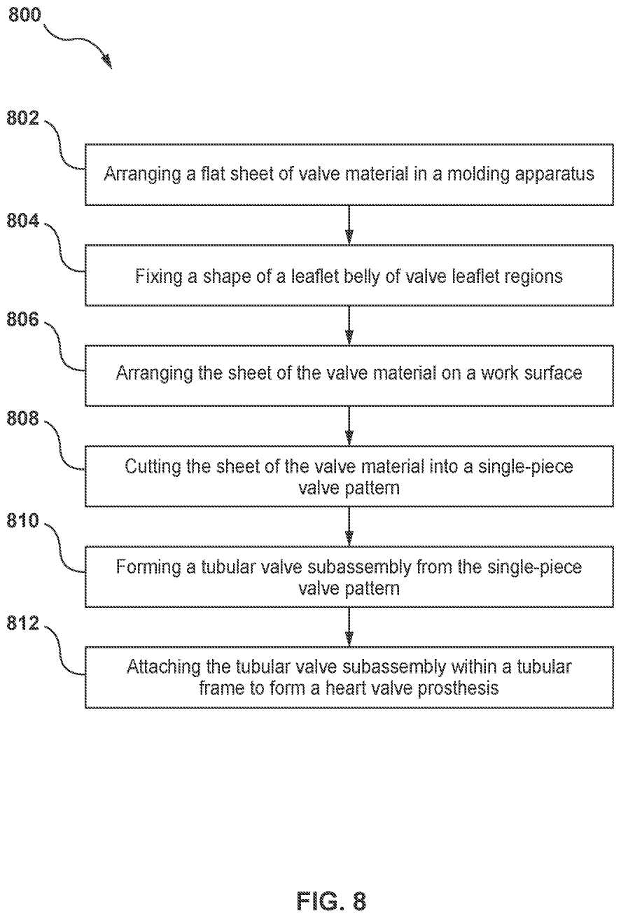

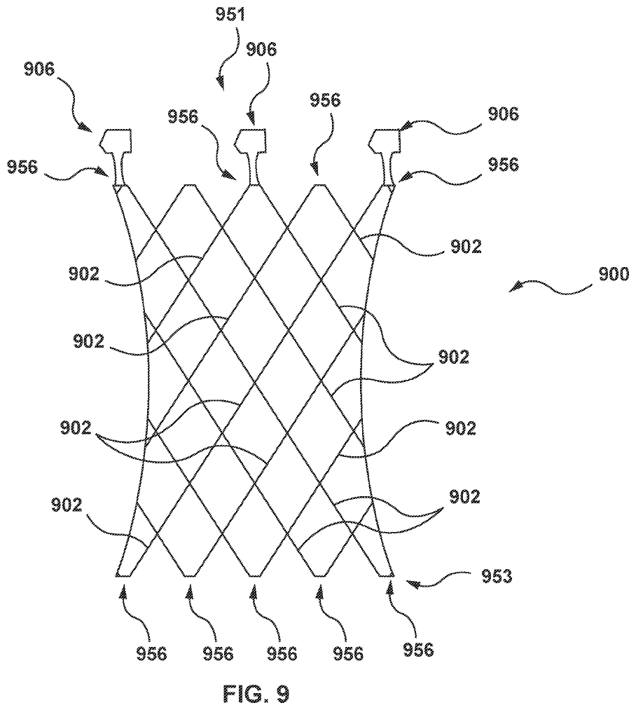

FIG. 8 illustrates an example of another method 800 for manufacturing a prosthetic heart valve, in accordance with an embodiment hereof. One skilled in the art will realize that FIG. 8 illustrates one example of a method that may be performed on the manufacturing system 100 and that existing operations illustrated in FIG. 8 may be removed and/or additional operations may be added to the method 800.

In step 802, a flat sheet of a valve material is arranged in a molding apparatus. In this method, a flat sheet 110 of a valve material 112 is composed of a material that can stretch and expand, for example, bovine pericardium tissue. Additionally, for example, the valve material 112 can be composed of polymers, e.g., polytetrafluoroethylene (PTFE), expanded polytetrafluoroethylene (E-PTFE), etc. Additionally, for example, the valve material 112 can be composed of woven fabrics, e.g., polyethylene terephthalate (PET), etc. Additionally, for example, the valve material 112 can be composed of electrospun materials, e.g., collagen, PET, etc. The molding apparatus 106 operates to shape portions of the valve material 112 into the leaflet bellies 230. For example, the bottom mold 502 and the top mold 504 can be separated thereby exposing the recesses 506 and the projection 510. The flat sheet 110 of the valve material 112 can be arranged in the molding apparatus 106 such that future valve leaflet regions 202, 204, 206 align between a respective recess 506 and its projection 510. For example, each of the valve leaflet regions 202, 204, 206 can be arranged within a recess 506. Once arranged, the bottom mold 502 and the top mold 504 are brought together thereby capturing the valve leaflet regions 202, 204, and 206 between the recesses 506 and the projections 510 and shaping the valve material 112 into the shape of the leaflet bellies 230.

In step 804, a shape of a leaflet belly of each valve leaflet region is fixed. In embodiments, to fix the shape of the leaflet bellies 230, the molding apparatus 106 is introduced to a fixing solution, such as a solution having glutaraldehyde, formaldehyde, or other fixative agent.

In step 806, the sheet of valve material having the leaflet bellies formed therein is arranged on a work surface. For example, as illustrated above in FIG. 1, the sheet of valve material having the leaflet bellies formed therein may be placed and arranged to be substantially flat on the top surface area 103 of the work surface 102.

In step 808, the sheet of valve material having the leaflet bellies formed therein is cut into a single-piece valve pattern. In embodiments, the cutting apparatus 108 can be activated to cut a single-piece valve pattern, such as the single-piece valve pattern 200 and/or 250. Because the valve leaflet regions 202, 204, 206 have already been shaped by the molding apparatus 106, the valve leaflet regions 202, 204, 206 include the leaflet bellies 230.

In step 810, a tubular valve subassembly is formed from the single-piece valve pattern. In step 812, the tubular valve subassembly is attached within a tubular frame to form the heart valve prosthesis. The tubular valve subassembly can be formed and attached to the tubular frame using processes similar to those described above in the method 600.

FIG. 9 illustrates an example of a tubular frame 900 that can be used with any of the embodiments disclosed herein. For example, the tubular frame 900 can be utilized as the tubular frame 750 in the methods 600 and 800 described above. One skilled in the art will realize that FIG. 9 illustrates one example of a tubular frame and that existing components illustrated in FIG. 9 may be removed and/or additional components may be added to the frame 900.

As illustrated in FIG. 9, the tubular frame 900 includes struts 902 formed in a diamond-shaped lattice structure. The struts 902 form crowns 956 at the first end 951 and the second end 953 of the tubular frame 900. Stated another way, the tubular frame 900 has a generally cylindrically-shaped mesh structure. In some embodiments, the tubular frame 900 can be self-expanding. For example, the tubular frame 900 can be constructed of a material that transitions from the compressed state to the uncompressed state at an implant side. For example, a tubular frame 900 can be formed from a shape memory material such as a nickel titanium alloy (e.g., Nitinol) that is self-expandable from the compressed state to the expanded state, such as by the application of heat, energy, and the like, or by the removal of external forces (e.g., compressive forces). In some embodiments, the tubular frame 900 can be expanded using expansion devices such as a balloon.

A prosthetic heart valve using the tubular frame 900 can be configured to replace or repair an aortic valve. Alternatively, other shapes are also envisioned, adapted to the specific anatomy of the valve to be repaired (e.g., stented prosthetic heart valves in accordance with the present disclosure can be shaped and/or sized for replacing a native mitral, pulmonic, or tricuspid valve).

In embodiments, the struts 902 arranged relative to each other to provide a desired compressibility and strength to the tubular valve subassembly. The tubular frame 900 can also include one or more paddles 904 positioned on one or more crowns 956 at the first end 951 for removably coupling a prosthetic heart valve including the tubular frame 900 to a delivery system. While FIG. 9 illustrate paddles 904, one skilled in the art will realize that the paddles 904 can be replaced with other components such as eyelets, loops, slots, or any other suitable coupling member. In embodiments, the tubular frame 900 includes an internal area or lumen in which a tubular valve subassembly in accordance herewith may be secured.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.

What is claimed is:

1. A method of manufacturing a heart valve prosthesis for transcatheter delivery comprising:
cutting a flat sheet of a valve material into a single-piece valve pattern having two or more valve leaflet regions and a valve skirt region;
arranging the two or more valve leaflet regions of the single-piece valve pattern into a mold for forming a leaflet belly for each of the two or more valve leaflet regions;
fixing, while arranged in the mold, a shape of the leaflet belly of each of two or more valve leaflet regions of the single piece-valve pattern;
creating a side seam by attaching two longitudinal edges of the single-piece valve pattern, to thereby form a tubular valve subassembly with two or more valve leaflets; and
attaching the tubular valve subassembly within a tubular frame to form a heart valve prosthesis.

2. The method of claim 1, wherein the single-piece valve pattern also includes an outer wrap region.

3. The method of claim 1, wherein cutting the flat sheet is performed with one or more of a laser or a cutting die.

4. The method of claim 1, the method further comprising:
creating a commissure on the tubular valve subassembly at each juncture between adjoining valve leaflets.

5. The method of claim 1, the method further comprising:
creating a margin of attachment for each of two or more valve leaflets of the tubular valve subassembly.

6. The method of claim 1, the method further comprising:
creating folds in the flat sheet of the valve material in each of the two or more valve leaflet regions, prior to performing the step of cutting.

7. The method of claim 7, wherein the folds provide for extra valve material in each of the two or more valve leaflet regions for creating 3-dimensional leaflet belly.

8. The method of claim 7, wherein creating the folds is performed by clamping the valve material in each of the two or more valve leaflet regions within adjacent arms of a folding device.

9. The method of claim 7, wherein creating the folds is performed by pulling the valve material in each of the two or more valve leaflet regions within a plurality of folding spaces of a folding device.

10. The method of claim 7, wherein creating the folds is performed by cinching the valve material in each of the two or more valve leaflet regions with a suture to create pleats.

11. The method of claim 7, wherein the folds are triangular shaped.

12. The method of claim 1, wherein the mold has a first part and a second part and the two or more valve leaflet regions of the single-piece valve pattern are inserted between the first and second parts of the mold to be sandwiched therebetween.

13. The method of claim 1, wherein creating the side seam is performed by sewing a thread or suture to join the two longitudinal edges of the single-piece valve pattern.

14. The method of claim 1, wherein attaching the tubular valve subassembly within the tubular frame is performed by sewing the tubular valve subassembly to the tubular frame with a thread or suture.

15. The method of claim 1, wherein the valve material is one of bovine or porcine pericardial tissue, a heat setting, synthetic material, or combination thereof.

16. A heart valve prosthesis comprising:
a tubular valve subassembly formed from a single-piece of valve material, the single-piece of valve material defining two or more valve leaflets and a valve skirt; and
a tubular frame,
wherein the tubular valve assembly is secured within an interior of the tubular frame.

17. The heart valve prosthesis of claim 16, wherein the single-piece of valve material further defines an outer wrap, and
wherein the outer wrap is secured on an exterior of the tubular frame.

18. The heart valve prosthesis of claim 16, wherein the valve material is one or more of biological tissue or synthetic material.

19. A system for forming a heart valve prosthesis, the system comprising:
a cutting apparatus configured to cut a flat sheet of a valve material into a single-piece valve pattern having two or more valve leaflet regions and a valve skirt region;
a molding apparatus configured to form a leaflet belly for each of the two or more valve leaflet regions, wherein the molding apparatus comprises at least two mating recesses and projections that form the valve material into a shape of the leaflet belly; and
a folding apparatus configured to hold folds in the flat sheet of the valve material in each of the two or more valve leaflet regions while the cutting apparatus cuts the flat sheet of the valve material.

20. The system of claim 19, wherein the folding apparatus comprises a folding device having a plurality of adjacent arms configured to clamp the valve material in each of the two or more valve leaflet regions.

21. The system of claim 19, wherein the folding apparatus comprises a folding device having a plurality of fixed tabs that form folding spaces.
